(19) United States
(12) Patent Application Publication
(10) Pub. No.: US 2002/0081750 A1
(43) Pub. Date: Jun. 27, 2002

(54) Drug evaluation operating principles

(76) Inventors: Michael Ernest, Rancho Santa Fe, CA (US); Doris L. Slate, Rancho Santa Fe, CA (US)

Correspondence Address: MCKENNA & CUNEO, LLP, 1900 K Street, NW, Washington, DC, 20006, US

(21) Appl. No.: 09/956,094

(22) Filed: Sep. 20, 2001

Related U.S. Application Data
(60) Provisional application No. 60/257,166, filed on Dec. 22, 2000.

(51) Int. Cl. C12Q 1/68; G01N 33/53; G01N 33/50; G01N 33/543; G06F 19/00

(52) U.S. Cl. 436/518; 435/7.1; 435/6; 702/19; 702/20

(57) ABSTRACT

The present invention relates to methods for determining whether a drug candidate should be advanced from discovery through evaluation to development and marketing. In one embodiment of the present invention, the drug development methods utilize a team decision-making format wherein scientific staff, and regulatory, financial, and marketing personnel may contribute to the evaluation of a new drug compound. In another embodiment of the methods of the present invention, decisions concerning the future of a potential drug may be made at earlier designated timepoints in the evaluation process, and these decisions may be made based on criteria such as preclinical pharmacological and toxicological data. In a further embodiment of the present invention, the potential new drug may be assigned a risk characterization, such as a color code, which defines the extent and duration of the evaluation process.

### CROSS REFERENCE TO RELATED APPLICATIONS

The present invention is related to and claims the benefit of, under 35 U.S.C. §119(e), U.S. provisional patent application Serial No. 60/257,166, filed Dec. 22, 2000, which is expressly incorporated fully herein by reference.

### FIELD OF THE INVENTION

This invention provides novel methods and processes for assessing and determining the advancement of a drug candidate from discovery through evaluation to development and marketing.

### BACKGROUND OF THE INVENTION

The typical path that the pharmaceutical industry follows in taking a product from discovery through evaluation to development and marketing may take as long as ten years, and may cost hundreds of millions of dollars. The process is generally divided into two stages: discovery and development. Discovery and identification of potential drug candidates are typically accomplished by several approaches: 1) traditional random screening of large numbers of compounds; 2) chemical modification of existing agents; 3) rational drug design based on known biological mechanisms and cellular targets; and 4) use of biotechnology methodologies. This last approach has revolutionized the pharmaceutical industry.

Biotechnology methodologies have produced an abundance of candidate drug targets. For example, molecular cloning techniques have lead to the discovery of hundreds of novel receptors and ion channels, as well as cytokines and other cell signaling proteins. Combinatorial chemistry and high-throughput screening provide the means to screen a vast number of compounds against numerous molecular targets. In addition, genomics, proteomics, DNA array technology, and bioinformatics offer other complementary approaches to identifying novel target candidates. These various techniques ensure that the research and development (R&D) pipeline is full of potentially successful pharmaceutical agents.

Following the discovery of potential therapeutic compounds, the biological activity of these compounds must be characterized and their pharmacologic profile defined. The activity and selectivity of a given compound are typically assessed by enzymatic activity assays, receptor binding assays, second messenger assays, whole cell and isolated tissue assays, and animal studies. The results of these studies assist in identifying the mechanism of action and the specificity of the candidate compound.

Once evaluated, the development stage for a test drug includes both preclinical and clinical studies. Promising candidates must also be evaluated for any possible toxicities. Preclinical toxicity testing includes acute toxicity, subchronic toxicity, chronic toxicity, reproductive and teratogenic effects, carcinogenicity, and mutagenicity. These in vivo animal studies are generally performed in mice, rats, dogs, and monkeys; some studies (e.g., chronic toxicity and carcinogenicity studies) may take up to 2 years to complete. In addition to toxicity effects, these studies are also utilized to estimate a maximum tolerated dose (MTD) which is used to calculate the initial dose to be administered in humans. Pharmacokinetic parameters such as systemic clearance, volume of distribution, plasma concentration, and half-life, may also be estimated from these preclinical studies.

Once a candidate has been deemed acceptable for testing in humans, and in order to obtain approval for sale of the drug in the United States, a Notice of claimed Investigational Exemption for a New Drug (IND) is filed with the U.S. Food and Drug Administration (FDA). An IND submission includes a clinical protocol, information on the chemistry and manufacture of the compound, and pharmacological and toxicological data from animal studies. The FDA requires that a proposed drug must pass through several phases of clinical trials before it can be marketed to the public. The clinical trials are generally divided into the three following phases:

Phase 1: In this phase, the clinical trials are typically open-labeled, dose-escalating studies in 25-50 healthy volunteers and usually last 4 to 9 months. In clinical trials for cancer or acquired immunodeficiency syndrome (AIDS) drugs, however, the volunteers are often patients suffering from the disease. The purpose of Phase 1 trials is to determine the safety of the drug in humans, human response to the drug, and dose-range limits. In addition, pharmacokinetic parameters may also be evaluated.

Phase 2: If no serious adverse reactions have been observed in Phase 1 clinical trials, then the study is expanded to include patients with the target disease. Phase 2 trials are separated into phase 2a and phase 2b. In Phase 2a trials, the efficacy of the test drug is determined, and biochemically relevant parameters and surrogate endpoints may also be assessed. These trials are usually of short duration (1-2 months) and typically 100-200 patients are enrolled. Drugs that prove to be efficacious and safe may be entered into Phase 2b trials. These trials are of longer duration (6-18 months) and they determine an optimal effective dose and dose interval, identify metabolites and other pharmacokinetic parameters of the test drug, and quantify any adverse reactions.

Phase 3: In Phase 3 trials, the safety and efficacy of the drug generally is further evaluated in large patient populations (i.e., several thousand patients). However, if serious adverse reactions or no significant benefits from the target drug have been observed in the patient groups from the Phase 2 trials, there may be no justification to pursue a Phase 3 study. To receive marketing approval from the FDA, the data from at least two Phase 3 studies must demonstrate the effectiveness of the drug for the proposed use. A New Drug Application (NDA) which includes all the preclinical and clinical data is submitted to the FDA. The decision by the FDA whether to approve the drug may take up to 3 years. Following approval and commercial distribution, postmarketing surveillance of adverse reactions of the drug is continued to ensure its safety.

As discussed above, pharmaceuticals are subject to extensive regulation by the FDA and the FDA must grant preliminary approval before any human studies may be conducted. Frequent reports are required for each phase study and, if unwarranted hazards to patients are observed, the FDA may request modification or discontinuation of clinical testing until further preclinical work has been performed. Depending on the target of the pharmaceutical, filing for FDA approval to market a drug may not be permitted until completion of either large-scale Phase 2 or Phase 3 trials. Because many foreign countries have shorter and/or less costly approval processes for new drugs, some members of the industry may seek to license and/or market its products in other countries prior to obtaining FDA approval.

The drug development methods described herein provide a novel approach to drug discovery, evaluation, and development. Typically, decisions concerning the developmental status of a new drug are not addressed until the clinical trial phase. However, a significant financial investment may have already been expended in a drug that may eventually prove to be toxic or an ineffective therapeutic. In addition, the current drug decision-making schemes utilized in the pharmaceutical industry are somewhat chaotic with limited communication between scientific staff, regulatory staff, and financial and marketing personnel. Thus, decisions concerning the future of a drug are often made with inadequate information and input from essential sources.

The methods of the present invention offer a streamlined decision-making process that ensures that the industry maintains a competitive edge while only the most effective and profitable drugs are developed. Unlike the current decision-making processes used by pharmaceutical companies, the drug development methods of the present invention preferably utilize a team decision-making format where the scientific staff, regulatory staff, and financial and marketing personnel contribute to the decision-making process. In addition, decisions concerning the future of a drug candidate are preferably made early in the development process, e.g., decisions are made based on preclinical pharmacological and toxicological data.

### SUMMARY OF THE INVENTION

In a specific embodiment, the present invention provides methods for selecting a new drug compound for advancement to drug development comprising the steps of discovering a new drug candidate for evaluation (NCE), selecting the NCE for drug evaluation, and evaluating the NCE for advancement into the new drug development stage. If the NCE passes the evaluation step, then a further embodiment of the present invention may include selecting the NCE as a drug development candidate and developing the drug development candidate.

In one embodiment of the method of the invention herein, the discovery step may include identifying a therapeutic target for the NCE. The therapeutic target may be identified using one or more techniques such as, for example, genomics, bioinformatics, proteomics, and combinatorial chemistry. The therapeutic target may include, for example, an enzyme, receptor, protein, nucleic acid or ion channel. In another embodiment of the method, the discovery step may include, for example, performing high-throughput screening and DNA array technology.

In still another embodiment of the method, the discovery step may comprise conducting one or more studies such as preclinical pharmacology studies, preclinical metabolism studies, and preclinical toxicity studies. The preclinical pharmacology studies may include, for example, in vitro assays that are cell-free, cell/tissue based, or both for target versus comparators. Alternatively, the preclinical pharmacology studies may include in vivo assays, including animal pharmacology for target versus comparators. Such in vivo animal pharmacology studies may use, for example, a single-dosing regimen of the NCE, or may use a multiple-dosing regimen of the NCE.

In an additional embodiment of the invention, the discovery step may comprise preclinical metabolism studies, such as evaluating key pharmacokinetic parameters. These pharmacokinetic parameters may include, for example, peak plasma concentration (Cₘₐₓ), Tₘₐₓ, half-life (t_{½}), bioavailability, and clearance parameters. These parameters may be used to develop human pharmacokinetic and pharmacodynamic (PK/PD) modeling of the candidate drug. Additionally, these parameters may be used to facilitate dose selection for toxicology analysis.

In another aspect of the invention, the preclinical metabolism studies may include in vitro metabolism studies in systems such as, for example, animal and hepatic S9 cells, liver microsomes, and hepatocytes. The preclinical metabolism studies may also be conducted in vivo, in single- and/or multi-dose pharmacokinetic/pharmacodynamic studies. Additionally, these in vivo studies may be used to identify and/or characterize major in vivo metabolites of the NCE.

Another embodiment of the invention may include a discovery step based on preclinical toxicity studies, such as in vitro studies that determine if the NCE exhibits disqualifying properties. Such disqualifying properties may be identified by an Ames test, with or without metabolic activation. Other preclinical toxicity studies used in the methods of the invention may include in vitro screening techniques such as receptor/enzyme/ion channel screening.

Still other preclinical toxicity studies of the present invention may include in vivo studies that indicate whether the NCE exhibits overt disqualifying properties. Such in vivo toxicity studies may include general cardiovascular (CV) and/or central nervous system (CNS) pharmacology evaluation in animals such as rodents and/or dogs. These studies may also comprise a three to five day toxicity test in rodents, wherein the test may include administering the NCE in a range about three to ten times higher than efficacious doses.

In a preferred embodiment of the invention, the method for advancing the NCE to the development stage may include the step of selecting the NCE for evaluation. The selecting step may include assessing the biological data obtained from one or more of the preclinical pharmacology, preclinical metabolism, and preclinical toxicity studies.

This selecting step may further comprise assessing information on the background, chemical data, laboratory synthetic scheme, patent status, and/or clinical data on the NCE. In one embodiment of the invention, the background information may include marketing opportunities for the NCE, which may include an assessment of the market competition for the NCE. In another aspect of the invention, the chemical data assessed in the selecting step may refer to physicochemical properties of the NCE. In another aspect of the invention, the clinical data assessed in the selecting step may also include projected clinical dose ranges, projected clinical plasma levels, and/or recommended surrogate markers of clinical efficacy based on preclinical findings.

In a preferred embodiment of the invention, the step of selecting the NCE may further comprise assigning a risk level for the NCE. The assigning step may further comprise defining the extent of evaluation of the NCE. The assigning step may also comprise defining the duration of the evaluation of the NCE.

The risk levels that may be assigned to the selected NCE include low risk, medium risk, and high risk levels. A low risk level may be assigned to an NCE having a well-defined therapeutic target. A particular aspect of the low risk NCE may be that the therapeutic target is linked to a clinically validated mechanism of action. In contrast, a medium risk level may be assigned to an NCE having a link between a therapeutic target and a mechanism of action. This link, however, may not be fully validated. Alternatively, a high risk level may be assigned to an NCE having a therapeutic target not yet defined for the mechanism of action.

Another aspect of the invention comprises the step of evaluating the selected NCE. This evaluation step may comprise conducting Phase 1 trials and/or Phase 2a trials in humans. In one aspect of the invention, the Phase 1 trial may include pharmacokinetic studies. In another aspect, this Phase 1 trial may include pharmacodynamic studies. The Phase 2a trials of the evaluation step may include establishing a dosing regimen for the NCE. Another aspect of the Phase 2a trials may comprise establishing an endpoint validation for the NCE.

A specific embodiment of the methods of the present invention may further comprise the step of selecting an evaluated NCE as a development candidate; in a preferred embodiment, the present invention may further include the step of developing the development candidate. This developing step may comprise establishing a commercial formulation for the development candidate. The developing step may also include performing further Phase 2a and/or Phase 2b clinical trials with the development candidate. In another aspect of the invention, the development step may comprise establishing a protocol for Phase 3 trials. In a preferred embodiment, a Phase 3 trial may be performed.

The developing step may preferably include filing an application for registration of the development candidate with the regulatory authority of a foreign jurisdiction. More preferably, the developing step may include filing for FDA approval to market the development candidate. Most preferably, the foreign filing may be done in addition to filing for registration in the United States. Such filing may be done after Phase 2 or Phase 3 clinical trials.

The evaluating step of the present invention preferably may include assembling a Drug Evaluation Core Team, a Global Project Team, a Global Pharmaceutical Strategic Marketing Team, and a First-in-Human Committee. The Drug Evaluation Core Team preferably may comprise scientists, clinicians, regulatory personnel, financial personnel, and marketing personnel. The Global Project Team preferably may comprise a global medical leader, Chemistry, Manufacturing, and Control (CM&C) and PCD leaders, a project director, and a commercial product team leader.

A preferred embodiment of the selecting step of the method of the present invention may comprise a decision to determine whether a drug should enter the evaluation stage by the Drug Evaluation Core Team, for example, within about one week after presentation by the drug discovery team.

In another embodiment of the present invention, the evaluation step may comprise defining the evaluation criteria and developing a draft clinical plan by the Drug Evaluation Core Team and the drug discovery team, for example, within two weeks after acceptance into the drug evaluation stage.

### BRIEF DESCRIPTION OF THE DRAWINGS

FIG. 1 is a graphical depiction of one embodiment of the present invention, in which effective drug evaluation bridges the gap between drug discovery and drug development. The methods described herein advances drug candidates through preclinical evaluation and into early clinical trials to investigate drug safety, efficacy, and delivery.

FIG. 2 illustrates a risk characterization for an NCE. In this embodiment, a red color-code may be assigned to an NCE where the mechanism of action for the therapeutic target has yet to be defined and is therefore considered a high risk NCE. A yellow color-code may be assigned to an NCE where the link between the therapeutic target and the mechanism of action is not yet fully validated and is therefore considered a medium risk NCE. A blue color-code may be assigned to an NCE where the well-defined therapeutic target linked to a clinically validated mechanism of action has been identified and is therefore considered a relatively low risk NCE.

FIG. 3, in a particular embodiment of the present invention, depicts a timeline of the involvement of the management bodies that exercise decisions based on data obtained from the drug evaluation program as the target drug passes from NCE to the development-candidate status. Specifically, the figure describes the growth of the management groups, designated Drug Discovery Team (DDT), the Drug Evaluation Core Team (DECT), the Global Project Team (GPT), the Global Commercialization Team (GCT), the First-in-Human Committee, and the Development Commitment Committee, as the NCE passes through the drug evaluation program and approaches each decision point for its continued development.

### DETAILED DESCRIPTION OF PREFERRED EMBODIMENTS

Before the present invention is described, it is to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “a drug” is a reference to one or more drugs and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated by reference for the purpose of describing and disclosing, for example, the methodologies that are described in the publications which might be used in connection with the presently described invention. The publications are provided solely for their disclosure prior to the filing date of the present application. Nothing herein is to be construed as an admission that the inventors are not entitled to antedate such disclosure by virtue of prior invention.

New technologies in the combinatorial chemistry, genomics, and bioinformatics areas have provided the pharmaceutical industry with the means to discover an unprecedented number of novel drug candidates. With these advances follows the reality that a highly selective and cost-efficient method is needed to identify and timely develop the most promising candidates. Indeed, once a promising drug candidate is found, a pharmaceutical company must contend with the ever-increasing cost of preclinical and clinical trials, particularly Phase 3 trials, which can be quite expensive due to the large patient population and volumes of data that must be collected and analyzed.

The methods of the present invention provide the means to maximize economic efficiency, maintain cost containment measures, reduce time to market; yet still permit a pharmaceutical company to be the first to market a new drug and thus, protect its financial investment. Indeed, the methodologies of the present invention allow pharmaceutical companies to evaluate the efficacy and safety data, as well as the financial promise, of a candidate drug before initiating costly long-term patient trials. This scheme preferably overlaps various candidate drug evaluation steps to maximize the amount of meaningful data gathered over a shorter time period and preferably provides defined timepoints at which a company can determine whether to continue development of a particular drug.

During the drug discovery stage of the methods described herein, specific criteria are preferably established to define areas of research that may be pursued. For example, the focus of drug development may be in the fields of cardiovascular disease, neurological disorders, or cancer. Next, specific target diseases for drug development, such as prostate cancer or Alzheimer's disease, may be defined. In addition, target effects and surrogate markers for these diseases may also be established. The decisions delineating the research objectives may be made, in a particular embodiment of the present invention, by a Drug Discovery Committee (DDC). The DDC members may include scientists, clinicians, management, and finance and marketing personnel, and the criteria that the DDC may utilize to identify research areas and target diseases may include market potential, competitor presence, resource and regulatory requirements, freedom to operate, and patentability. Establishing one or more of these criteria early on in the drug discovery and development process, in the context of the methods of the present invention, preferably maximizes resources in order to pursue promising drugs and thus minimize the risk of investing in ineffective, and thus unprofitable, drugs.

As an example, to assess the presence of a competitor in the pharmaceutical market, several factors may be considered such as capabilities and strategies of a competitor, probable future actions or goals, price, product features and functions, and consumer perception of the competitor. In addition, market potential may be evaluated by determining who might be the most likely consumer, that is, who is willing and able to purchase a product. Thus, marketers may look to consumer interest and income, market risk and saturation, and market share. With respect to patentability, a product must meet the requirements set out under U.S. patent law (i.e., statutory subject matter, usefulness, novelty, and nonobviousness) in order to qualify for patent protection. Furthermore, a company may need to secure its freedom to operate to avoid any potential patent infringement issues. In particular, it may be necessary to license additional technologies necessary to launch a product or develop alternative techniques. Therefore, in addition to drug efficacy, a pharmaceutical company may need to consider a number of other criteria before pursuing the development of a new drug.

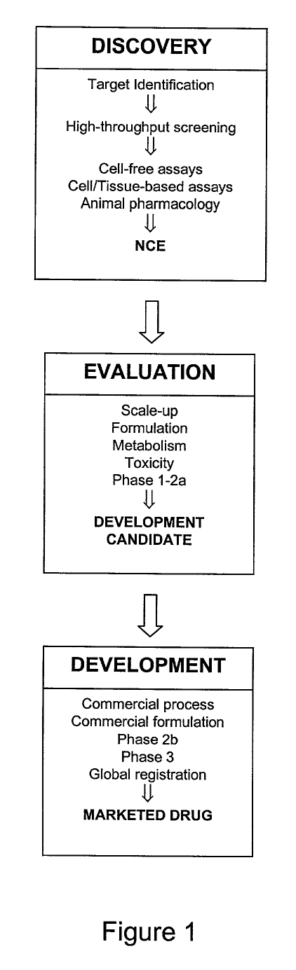

The drug evaluation methods described herein provide an efficient method to advance candidate drugs (NCEs) through preclinical evaluation and into early clinical trials to investigate drug safety, efficacy, and delivery. For example, as illustrated in FIG. 1, a specific disease target such as Alzheimer's disease may be selected by the DDC, and using biotechnology methods (e.g., high-throughput screening or DNA array technology), several novel compounds may be identified as potential therapeutics for the target disease. The pharmacological characteristics of these compounds may then be analyzed utilizing tissue-based assays or animal studies. If a compound demonstrates a promising pharmacological profile, it may then be designated as an NCE. The NCE may then advance into the evaluation stage where numerous criteria such as scale-up and formulation costs or the toxicity effects observed in Phase 1 trials, may be evaluated to determine whether the NCE should be considered a development candidate. If the NCE is deemed eligible following the evaluation stage, it then proceeds to the drug development stage. During this stage, Phase 2b or Phase 3 trials may be initiated; commercialization issues may be addressed; and registration with the FDA or other international agencies may be pursued, eventually leading to the marketing of the NCE.

One aspect of the drug evaluation methods of the present invention preferably is to reduce some of the inherent financial risk in drug discovery and development; effective drug evaluation that bridges the gap between the traditional discovery and development stages accomplishes this. Implementation of the drug evaluation methodologies of the present invention permits more informed decisions regarding the entry of NCEs into the development portfolio. In turn, a company may focus resources on those compounds presenting the best opportunity for successful world-wide registration and marketing. Factors influencing risk assessment of a candidate drug may include low potency or lack of efficacy; poor pharmacokinetics (short half-life, low bioavailability); acute or subchronic toxicity; and narrow therapeutic index.

The drug development methods of the present invention provide a superior alternative to current ad hoc methodologies, because the steps are logically defined and preferably implemented in an organized fashion. The steps include the drug discovery process itself, which concludes with the identification of NCEs. If the discovery process does not yield a favorable pharmacological profile for a candidate drug, the drug may never enter the more rigorous evaluation program. This decision may balance the data gained from the discovery process against the financial risk associated with further study. If, however, the discovery steps provide promising information supporting the NCE, it may then enter the drug evaluation program, thus providing further information on whether the drug should become a candidate for further development.

At this point in time, the extent of the evaluation process may be determined by assigning the NCE a risk level. The criteria implemented to analyze and assess information during the evaluation process may be specifically defined by a Drug Evaluation Core Team (DECT). If the information obtained in the evaluation process warrants, the NCE may become a drug development candidate, for which further research may be conducted as the development candidate nears actual marketing. This approach may also be followed for NCE candidates that are obtained from outside sources. The drug evaluation operating principles are described as follows.

Biological information that defines the drug candidates is assembled during the drug discovery phase. This information may include pharmacological data such as, for example, cell-free activity of the target enzyme, receptor, or ion channel vs. comparators; cell- or tissue-based assays vs. comparators; animal pharmacokinetic activity vs. comparators during single- and multiple-dosing regimens. In addition, metabolic activity data including, for example, animal and human hepatic S9 cells, liver microsomes, or hepatocyte metabolism in vitro; single- and multiple-dose pharmacokinetics in pharmacology and/or toxicology species; and preliminary identification and characterization of major in vivo metabolites; and toxicology data such as, for example, screening Ames test ±metabolic activation; receptor/enzyme/ion channel screening; general cardiovascular (CV) and central nervous system (CNS) pharmacology evaluation in rodents and/or dogs; and three- to five-day toxicity in rodents at three to ten times efficacious doses in animal tests may also be included in the drug characterization profile. A Drug Discovery Team (DDT) may be responsible for compiling this information in anticipation of the Drug Evaluation procedure.

The preclinical pharmacology analyses may begin with a determination of the activity of the drug candidate in the isolated human molecular target and then progress through increasing biological complexity to animal models (see e.g., GILMAN et al., GOODMAN AND GILMAN'S THE PHARMACOLOGICAL BASIS OF THERAPEUTICS, Macmillan Publishing Co., NY, 1985; Berry et al., 51 LIFE SCI. 1-16,1992). Cell- or tissue-based assays should use primarily human material, if available, in addition to the appropriate animal samples for subsequent in vivo studies. Animal pharmacology, in the methodologies of the present invention, may focus on one or two features that mimic human disease pathology, rather than the typical approach which uses extensive animal model development and testing. The emphasis here is on surrogate markers of efficacy (biochemical or pharmacological) that can be extended into the clinic (see e.g., Prentice, 8 STAT. MED. 431-440, 1989).

The preclinical metabolic assays may evaluate key pharmacokinetic parameters (e.g., Cₘₐₓ, Tₘₐₓ, t_{½}, bioavailability, and clearance) in pharmacology and/or toxicology species (mice, rats, and dogs) that enable human pharmacokinetic (PK) and pharmacodynamic (PD) modeling and facilitation of dose selection for clinical trials (see e.g., Chiu, 29 J. PHARMACOL. TOXICOL. METHODS 77-83, 1993; Dogterom, 21 DRUG METAB. DISPOS. 699-704, 1993; Guillouzo et al., 82 TOXICOLOGY 209-19, 1993; Houston, 47 BIOCHEM. PHARMACOL. 1469-79, 1994; Remmel and Burchell, 46 BIOCHEM. PHARMACOL 559-66, 1993; Rodrigues, 48 BIOCHEM. PHARMACOL. 2147-56, 1994).

The preclinical toxicity studies may use a series of non-GLP (Good Laboratory Practice), in vitro and in vivo studies that determine if the NCE shows overt, disqualifying properties prior to formal GLP toxicology during drug evaluation (see e.g., Berry et al., 1992; Clark and Smith, 12 CRIT. REV. TOXICOL. 343-85, 1984; Powis, 20 DRUG METAB. REV. 379-94, 1989; Wrighton and Stevens, 22 CRC CRIT. REV. TOXICOL. 1-21, 1992).

The biological data from the NCE studies may be compiled, for example, into a Compound Monograph by the DDT. This monograph may contain, for example, an introductory background section describing opportunity and competition for the NCE; a chemistry section summarizing the physicochemical properties of the NCE; current laboratory synthetic scheme and patent status; and a clinical section including projected clinical dose range/plasma levels and recommended surrogate markers of clinical efficacy based on preclinical findings. The monograph may also be used as the initial version of the Investigator's Brochure in the IND filing with the FDA.

The DECT may then determine and select which compounds shall pass from the NCE stage to the Evaluation stage. The DECT may, for example, be a self-managed group composed of senior scientists from the various clinical and non-clinical functions, regulatory affairs, and strategic marketing groups. These may include personnel, for example, representing discovery, process chemistry, metabolism, formulation, regulatory, clinical, and strategic management. The final decision may be the responsibility of the Vice President for Drug Evaluation, or an officer of similar position, who may also inform the management boards of the company on the progress of all compounds in drug evaluation.

In a particular embodiment, the sponsoring DDT presents the NCE's monograph to the DECT and associated functional area representatives. Preferably within, for example, about one week from submission, this group determines the status of the NCE. Acceptance by the group signals the entrance of the NCE into the evaluation process. Alternatively, if the NCE is denied entry into the evaluation stage, a written report may be furnished to the discovery team outlining deficiencies that must be addressed for reconsideration of the NCE at a later date.

In order to assess the NCE, the DECT may review the results of the preclinical in vitro and in vivo studies. These studies provide a working pharmacological and toxicological profile for the NCE. The DECT may evaluate a number of factors such as, for example, the validity and accuracy of the preclinical studies; the target pharmacological response; toxicity effects; and pharmacokinetic parameters including serum concentrations, absorption, distribution, and elimination. The data from these studies may be used to determine whether administration of the NCE to humans is feasible.

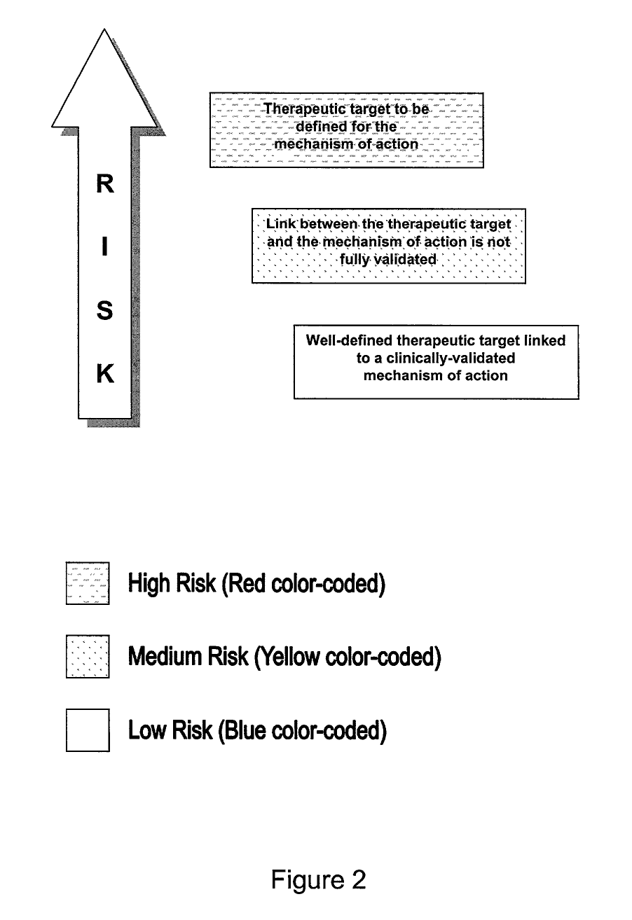

Additionally, the review entity may assign the NCE a risk characterization such as a color code which may reflect, for example, the extent and duration of the evaluation program, the content of the target core data sheet, and the trigger point for the creation of “shadow” global project and commercial teams. For example, an NCE for which the mechanism of action for the therapeutic target has yet to be defined may be considered a high risk NCE that gets color-coded Red, as shown in FIG. 2. An NCE for which the link between the therapeutic target and the mechanism of action is not yet fully validated may be considered medium risk, and color-coded Yellow. Finally, an NCE for which the well-defined therapeutic target linked to a clinically validated mechanism of action has been realized may be considered a comparatively low risk NCE that gets color-coded Blue.

Although the mechanism of action of “low risk” (i.e., “blue”) NCEs preferably have been clinically validated for target diseases, these compounds may still be risky with respect to potential and undesirable clinical (and non-clinical) safety and metabolism findings, as well as to scale-up and formulation challenges. Therefore, rather than move these compounds directly into development with attendant visibility, staffing, and documentation needs, it may be desirable to conduct a focused evaluation program to eliminate some of the up-front risk. Because it is likely that validated surrogate markers predictive of clinical efficacy will be available, along with the relevant comparators, the focused evaluation program may be accomplished in one or two Phase 1 trials on healthy subjects. At the same time that a low-risk compound enters Drug Evaluation, a “shadow” development group (e.g., a Global Project Team (“GPT”)) may be appointed to prepare for a timely full development decision on the NCE.

Following acceptance of an NCE into drug evaluation, and preferably within two weeks after acceptance, the evaluation criteria may be defined, preferably in collaboration with the DDT, and may be used to draft a clinical plan. Depending on the mechanism of action and the potential disease targets, evaluation of the NCE may end after Phase 1 (when validated surrogate markers of clinical outcome are available), or the evaluation may extend into preferably small, focused Phase 2a studies in patients. The clinical evaluation plan preferably defines the toxicology/metabolism program to be conducted, which in turn will determine good manufacturing practice (GMP) and non-GMP drug substance and product requirements. Once these plans and requirements are set in place, a goal is to execute the program at the functional level, preferably with a mix of dedicated and ad hoc internal and external personnel.

The Phase 1 and 2a clinical trials may be designed to maximize the collection of relevant pharmacological and toxicological data, and to minimize extraneous information. For example, patient population, dosage, dosing interval, duration, and mode of administration preferably are carefully selected to achieve the study objectives. Smaller scale studies with rapid patient recruitment may be developed to provide necessary information regarding the safety and efficacy of the candidate drug. If these clinical trials demonstrate that the candidate drug is safe and effective, then Phase 2b or 3 trials may be initiated. Conversely, if serious toxic effects are observed or the candidate drug proves to be ineffective in humans, then drug development may be terminated, thereby avoiding costly Phase 3 trials.

The evaluation criteria may also include results from these planned clinical “proof-ofprinciple” and non-clinical “proof-of-principle” trials. These results may be regularly evaluated against the prospectively defined evaluation criteria. If a compound continues to meet these criteria, then the additional research needed to reach the full development decision point may continue. If a compound does not meet the specified targets, further work on the compound may be terminated at a logical stopping point. In some instances, an NCE that is unlikely to become a full development candidate may continue through evaluation in order to prove principle in the clinic, prepare the compound for out-licensing, or establish the pathway for backup compounds.

As mentioned previously, and prior to the end of the evaluation and a full development decision, a shadow GPT may be created for compounds that are likely to meet the drug evaluation criteria. A shadow GPT may include, for example, a global medical leader; CM&C leader; and a project director (all appointed from the R&D organization), as well as a commercial product team leader or designate from the global pharmaceutical strategic marketing (shadow global commercial team) department. Participants on these shadow teams may be responsible for preparing the initial versions of the target core data sheet, and/or financial and other analyses necessary to justify bringing the compound into the development portfolio. In a specific embodiment, the members of the shadow teams may go on to become the core members of the GPT and the Global Commercial Team (GCT) when and if the compound enters full development.

Upon successful completion of the preclinical portion of the methodologies of the present invention, the DECT may recommend initiation of clinical trials to a First-in-Human (FIH) Committee. The FIH Committee may consist of, for example, scientific members of the management boards of the operating companies, who review the scientific, regulatory, and ethical aspects of the clinical evaluation program in the context of the first human trial. The FIH may either approve initiation of the clinical program as proposed, approve with revisions and/or requirements to be completed before trial initiation, or disapprove with cause.

The full development decision may follow completion of the clinical evaluation. The DECT, the GPT, and the GCT may present jointly the results of the evaluation program, the proposed development program, and the portfolio analysis to the appropriate management board (of the operating company that will develop and register the drug) and to the Development Commitment Committee (DCC) with a recommendation for full development, out-licensing, or termination.

The development candidate may then enter the development phase. This may entail establishing a commercial formulation for the development candidate and may require planning and perhaps conducting further Phase 2a and/or Phase 2b clinical trials with the development candidate. At this point in the development process, it may be appropriate to invest in the establishment of a protocol for Phase 3 trials as well. The scale-up to commercial processing levels may also be appropriate at this time.

Additionally, the selection of the development candidate may also signal the appropriate time for the filing of applications for registration of the drug with the regulatory authority of target markets. This may include both domestic and foreign authorities. In particular, development of the candidate drug may include filing for FDA approval to market the drug and may occur after Phase 2 or Phase 3 clinical trials.

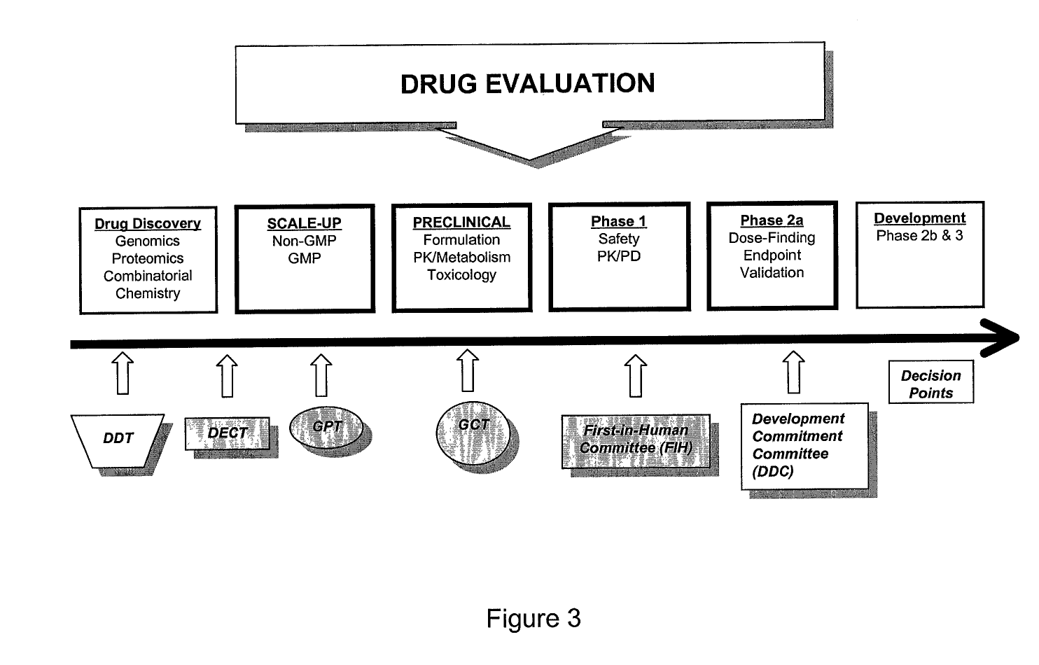

An explanatory timeline of the involvement of the management bodies that exercise decisions based on data obtained from the drug evaluation program as the target drug passes from NCE to the development-candidate status is illustrated in FIG. 3. Briefly, the DDT may employ technologies such as genomics, combinatorial chemistry, and pharmacological assays to identify and characterize novel drug candidates. The DDT then may prepare a Monograph describing these studies and may present the data to the DECT. In turn, the DECT may consider this data as well as other information including scale-up costs. If the data gathered for the drug candidate is deemed acceptable, then the drug may be classified as an NCE. During the drug evaluation stage, supplementary studies such as metabolic and toxicological studies may be performed for further characterization of the NCE. In addition, a GPT and a GCT may be created to participate in the decision-making process. Following an evaluation of the preclinical data, Phase 1 studies may be initiated and an FIH committee may be assembled to design the clinical trials. If the NCE proves to be safe, Phase 2a trials may be performed to establish efficacy, dosages, and endpoint validation. The results of this drug evaluation may then be presented to the DCC with a recommendation for full development.

The methods of the present invention offer a timely approach to drug development. One embodiment of the method of the present invention introduces the concept of team decision-making at earlier timepoints in the development process. Unlike the current decision-making process, whereby a limited number of executives are involved, the methods of the present invention utilize the combined resources of numerous disciplines such as scientists and clinicians, as well as regulatory, financial, and marketing personnel. All members of the various teams preferably contribute to decisions concerning target identification, evaluation criteria, clinical trial design, financial expenditures, and marketing potential. During the evaluation process of the method of the present invention, decisions regarding the future of candidate drugs preferably are made based on preclinical pharmacological and toxicity data. Drugs that do not demonstrate potential are quickly eliminated early in the development stage, while development of promising drugs is continued with reallocation of financial resources to promote success.

Without further elaboration, one skilled in the art can, using the preceding description, utilize the present invention to the fullest extent. Various modifications and variations of the described methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims. References are fully and specifically incorporated herein by reference.

### EXAMPLES

The following example is illustrative only, and not limiting of the remainder of the disclosure in any way whatsoever.

### Example 1

The search for a new pharmaceutical product begins with the identification of a specific target disease by the Drug Discovery Committee (DDC). To select a particular disease, the DDC considers a number of criteria including disease conditions, current commercially available drugs, the effectiveness of these drugs, the potential patient population, and the FDA requirements to register a new drug (e.g., required pharmacokinetic and toxicity studies). The DDC also performs a market survey to assess its market opportunities. In addition, the DDC also establishes surrogate markers and clinical endpoints for the target disease.

Based on its analysis, the DDC elects to focus on cardiovascular disease, in particular coronary artery disease (CAD). Generally, CAD is associated with myocardial infarction (MI) where the combination of atherosclerosis, platelet activation, thrombosis, and vasospasm result in the occlusion of a coronary vessel leading to an MI. In the U.S. as well as other countries, MI is the leading cause of death; each year approximately 1.5 million individuals suffer an MI and 500,000 people die (COTRAN ET AL., ROBBINS PATHOLOGIC BASIS OF DISEASE, W.B. Saunders Co., Philadelphia, Pa., 1999). One treatment option for atherosclerosis and CAD is the use of cholesterol-lowering drugs, specifically inhibitors of 3-hydroxy-3-methylglutaryl coenzyme A (HMG-CoA) reductase which is a key rate-limiting enzyme in cholesterol biosynthesis (MATHEWS AND VAN HOLDE, BIOCHEMISTRY, The Benjamin/Cummings Publishing Co., Redwood City, Calif., 1990). However, although treatment with cholesterol-lowering drugs has been somewhat successful, many patients continue to have clinical events (Superko, 2 CURR. TREAT. OPTIONS CARDIOVASC. MED. 173-87, 2000). Taking these factors into consideration, the DDC concludes that there is a It justification to develop a more effective cholesterol-lowering drug. As a surrogate marker for CAD, the DDC recommends a decrease in plasma cholesterol levels via an inhibition of HMG-CoA reductase and, as a clinical endpoint, a decrease in the occurrence of CAD.

Once the target disease is identified, a DDT is assembled to begin the drug discovery phase. Using high-throughput screening techniques, the DDT discovers a potential candidate that possesses the requisite inhibitory effect on HMG-CoA reductase activity. To further characterize the drug candidate, pharmacologic, metabolic, and toxicity studies are performed by the DDT. Specifically, in vitro cell culture and in vivo animal studies are conducted to assay for HMG-CoA reductase activity of the drug candidate (see e.g., Roullet et al., 90 PROC. NATL. ACAD. SCI. USA 11728-32, 1993; Rao et al., 96 PROC. NATL. ACAD. SCI. USA 7797-7802, 1999). For the metabolic studies, several parameters are analyzed including absorption, distribution, metabolism, and excretion of the drug candidate and pharmacokinetic factors such as volume of distribution (V_{d}), half-life (t_{½}), clearance (Cl), and bioavailability (F) are also determined (see e.g., Obach et al., 283 J. PHARMACOL. EXP. THER. 46-58, 1997; Lin, 26 DRUG MET. DISp. 1202-12, 1998). Toxicity studies are performed in three animal models (mouse, beagle dog, and rhesus monkey) using both single-dose and multiple-dose schedules, and data from clinical chemistry, hematology, histology, and necropsy studies are recorded.

The DDT compiles the data from the drug discovery studies into the Compound Monograph. In addition to the pharmacology and toxicology results, the monograph contains a chemistry section summarizing the physicochemical properties of the NCE, and a clinical section describing projected clinical dose ranges and plasma levels in humans. The monograph also includes information pertaining to current laboratory synthetic schemes and patent status. If the drug candidate is developed, the monograph will be used as the initial version of the Investigator's Brochure in the IND filing with the FDA.

The DDT then presents the Monograph to the DECT for review of the data from the drug discovery and preclinical studies. In this case, the drug candidate demonstrates a significant inhibition of HMG-CoA reductase activity in cell culture studies as well as a significant reduction in plasma cholesterol levels in animal studies meeting the surrogate marker requirement established by the DDC. Furthermore, the drug candidate exhibits excellent bioavailability, a reasonable half-life, sufficient clearance, and minimal toxic effects. Within one week following submission by the DDT, the DECT designates the drug candidate as an NCE signaling its acceptance into the drug evaluation process. Conversely, if the drug candidate did not, for example, inhibit HMG-CoA reductase activity, displayed a short half-life or poor absorption, or produced highly toxic effects, then the DECT would have denied entry into the drug evaluation stage. In addition, the DECT would have submitted a written report to the DDT delineating any study deficiencies or recommendations that would need to be addressed for reconsideration at a later date.

A risk characterization is assigned to the NCE by the DECT reflecting the extent and duration of the evaluation process. In this case, the therapeutic target (HMG-COA reductase) is well-defined and linked to a clinically validated mechanism of action. That is, the inhibition of HMG-CoA reductase activity as a means to treat CAD is a generally accepted method of therapy. Therefore, this NCE is deemed to be of relatively low risk and is color-coded blue. However, although this NCE is considered low risk with respect to its mechanism of action, other potential factors such as clinical toxic effects or exorbitant scale-up or formulation costs still warrant careful attention during the evaluation process and a focused evaluation program is initiated to address these issues. If the mechanism of action for this NCE had not been defined, then it would be considered a high risk NCE and would have been color-coded red. In that case, a comprehensive evaluation process would have been developed to address these high risk concerns.

Within two weeks after acceptance into the evaluation stage, the DECT, with input from the DDT, defines the criteria that is used to evaluate the NCE and also drafts a clinical plan for the NCE. The evaluation criteria defines anticipated costs for scale-up and formulation, GMP and non-GMP requirements, outsourcing or licensing of technologies necessary for drug production, in-house technology development, and patentability status.

At this time, a “shadow” GPT and a “shadow” GCT are created to fast track the NCE through the drug evaluation process. The GPT and GCT provide financial analyses of manufacturing, quality control, and technology costs, marketing strategies, and licensing opportunities.

For the clinical plan, the DECT designs a Phase I clinical trial scheme which maximizes data collection taking into consideration patient population, and the appropriate dosage and dose schedules based on the preclinical animal studies. In addition, the FDA has established guidelines for clinical trials and clinical evaluation of lipid-altering drugs such as an inhibitor of HMG-CoA reductase (GUIDELINES FOR THE CLINICAL EVALUATION OF LIPID-ALTERING AGENTS IN ADULTS AND CHILDREN, U.S. Food & Drug Administration, September, 1990) and thus, the DECT designs the clinical trials in compliance with these guidelines. To expedite drug evaluation, the clinical trials are small scale studies with rapid recruitment allowing for a timely assessment of the safety and efficacy of the NCE. The DECT then recommends initiation of clinical trials to the FIH Committee.

The FIH Committee reviews the preclinical data and proposed clinical trial design. In particular, the FIH examines the regulatory and ethical aspects of the trial design to determine whether the trial complies with the established FDA guidelines. Based on the promising preclinical data such as the significant inhibition of HMG-CoA reductase and lack of toxicity observed in animal studies, and acceptable trial design, the FIH approves initiation of clinical trials in human patients. The FIH may have denied approval if, for example, the trial design did not meet the requirements of the FDA guidelines, the preclinical data did not verify an inhibitory effect, or a harmful toxic effect had been noted.

Since the mechanism of action and a validated surrogate marker have been established for this NCE, the DECT elects to limit the drug evaluation of the NCE to two Phase 1 clinical trials. Following the completion of the clinical trials, the DECT, GPT, and GCT compile the results of the data accumulated during the drug discovery and evaluation stages and review the data to determine whether additional studies are needed before a decision can be made regarding the development status of the NCE. In this case, the NCE meets the criteria initially defined by the DDC. That is, a decrease in plasma cholesterol levels and minimal toxicity effects are observed in clinical trials. In addition, analysis of manufacturing costs, marketability, and patentability suggests that development of this NCE is a reasonable financial risk. Thus, the DECT, GPT, and GCT present the results of the evaluation process to the Development Commitment Committee (DCC) and recommend full development of this NCE.

If this NCE had partially met the evaluation criteria, then the DECT, GPT, and GCT may recommend that additional research is needed and a decision for full development would be made at a later time. If the NCE does not meet any of the specified targets, then full development would not be recommended and evaluation of the NCE would be terminated.

However, in some instances, an ineffective NCE may continue through the evaluation process in order to prove principle in the clinic, prepare the compound for out-licensing, or establish the pathway for backup compounds.

I claim:

1. A method for selecting a new drug compound for advancement to new drug development comprising the steps of:
discovering a new drug compound (NCE);
selecting said NCE for drug evaluation; and
evaluating said NCE for advancement into new drug development.

2. The method of claim 1, wherein said discovering step comprises identifying a therapeutic target for said NCE.

3. The method of claim 2, wherein said therapeutic target is identified by utilizing one or more of the techniques selected from the group consisting of genomics, bioinformatics, proteomics, and combinatorial chemistry.

4. The method of claim 2, wherein said therapeutic target comprises an enzyme, receptor, protein, nucleic acid, or ion channel.

5. The method of claim 1, wherein said discovering step comprises performing high-throughput screening.

6. The method of claim 1, wherein said discovering step comprises analyzing the efficacy of a drug by performing DNA array technology.

7. The method of claim 1, wherein said discovering step comprises one or more studies selected from the group consisting of preclinical pharmacology studies, preclinical metabolism studies, and preclinical toxicity studies.

8. The method of claim 7, wherein said preclinical pharmacology studies comprise in vitro assays.

9. The method of claim 7, wherein said preclinical pharmacology studies comprise in vivo assays.

10. The method of claim 8, wherein said in vitro assays comprise cell-free assays for target versus comparators.

11. The method of claim 8, wherein said in vitro assays comprise cell or tissue based assays for target versus comparators.

12. The method of claim 9, wherein said in vivo assays comprise animal pharmacology for target versus comparators.

13. The method of claim 12, wherein said animal pharmacology is performed using a single-dosing regimen of said NCE.

14. The method of claim 12, wherein said animal pharmacology is performed using a multiple-dosing regimen of said NCE.

15. The method of claim 7, wherein said preclinical metabolism studies comprise evaluating key pharmacokinetic parameters.

16. The method of claim 15, wherein said parameters are selected from the group consisting of Cₘₐₓ, Tₘₐₓ, t_{½}, bioavailability, and clearance.

17. The method of claim 15, wherein said parameters are used to develop human pharmacokinetic/pharmacodynamic (PK/PD) modeling.

18. The method of claim 16, wherein said parameters are used to facilitate dose selection for toxicology.

19. The method of claim 7, wherein said preclinical metabolism studies comprise in vitro metabolism studies in one or more systems selected from the group consisting of animal and human hepatic S9 cells, liver microsomes, and hepatocytes.

20. The method of claim 7, wherein said preclinical metabolism studies comprise preliminary identification of major in vivo metabolites.

21. The method of claim 7, wherein said preclinical metabolism studies comprise preliminary characterization of major in vivo metabolites.

22. The method of claim 7, wherein said preclinical metabolism studies are performed using single-dose pharmacokinetics in pharmacology species.

23. The method of claim 7, wherein said preclinical metabolism studies are performed using multiple-dose pharmacokinetics in pharmacology species.

24. The method of claim 7, wherein said preclinical toxicity studies comprise in vitro studies.

25. The method of claim 24, wherein said in vitro studies comprise determining if said NCE exhibits disqualifying properties.

26. The method of claim 24, wherein said in vitro studies comprise an Ames test with metabolic activation.

27. The method of claim 24, wherein said in vitro studies comprise an Ames test without metabolic activation.

28. The method of claim 24, wherein said in vitro studies comprise performing one or more screening techniques selected from the group consisting of receptor, enzyme, or ion channel screening.

29. The method of claim 7, wherein said preclinical toxicity studies comprise in vivo studies.

30. The method of claim 29, wherein said in vivo studies comprise determining if said NCE exhibits overt, disqualifying properties.

31. The method of claim 29, wherein said in vivo studies comprise a general CV and CNS pharmacology evaluation in rodents.

32. The method of claim 29, wherein said in vivo studies comprise a general CV and CNS pharmacology evaluation in dogs.

33. The method of claim 29, wherein said in vivo studies comprise a three to five-day toxicity test in rodents.

34. The method of claim 33, wherein said toxicity test comprises administering said NCE in a range about 3 to 10 times the efficacious doses in said rodents.

35. The method of claim 1, wherein said selecting step of said NCE advances said NCE from said discovering to said evaluating step.

36. The method of claim 35, wherein said selecting step comprises assessing biological data generated from one or more studies selected from the group consisting of preclinical pharmacology, preclinical metabolism, and preclinical toxicity.

37. The method of claim 36, wherein said selecting step further comprises assessing information from one or more of the group consisting of background information of said NCE, chemical data of said NCE, current laboratory synthetic scheme of said NCE, patent status of said NCE, and clinical data of said NCE.

38. The method of claim 37, wherein said background information comprises opportunity in the marketplace for said NCE.

39. The method of claim 37, wherein said background information comprises competition in the marketplace for said NCE.

40. The method of claim 37, wherein said chemical data comprises the physicochemical properties of said NCE.

41. The method of claim 37, wherein said clinical data comprises projected clinical dose ranges.

42. The method of claim 37, wherein said clinical data comprises projected clinical plasma levels.

43. The method of claim 37, wherein said clinical data comprises recommending surrogate markers of clinical efficacy based upon preclinical findings.

44. The method of claim 1, wherein said selection step comprises assigning a risk level for said NCE.

45. The method of claim 1, wherein said evaluation step comprises the steps of:
conducting Phase 1 trials in humans; and
conducting Phase 2a trials in humans.

46. The method of claim 44, wherein said assigning step further comprises defining the extent of the evaluation program for said NCE.

47. The method of claim 44, wherein said assigning step further comprises defining the duration of the evaluation program for said NCE.

48. The method of claim 44, wherein said assigning step comprises assigning a low risk level.

49. The method of claim 48, wherein said assigning a low risk level is for an NCE having a well-defined therapeutic target.

50. The method of claim 49, wherein said therapeutic target is linked to a clinically validated mechanism of action.

51. The method of claim 44, wherein said assigning step comprises assigning a medium risk level.

52. The method of claim 51, wherein said assigning a medium risk level is for an NCE having a link between a therapeutic target and mechanism of action.

53. The method of claim 52, wherein said link is not fully validated.

54. The method of claim 44, wherein said assigning step comprises assigning a high risk level.

55. The method of claim 54, wherein said assigning a high risk level is for an NCE having a therapeutic target with an undefined mechanism of action.

56. The method of claim 45, wherein said Phase 1 trial comprises pharmacokinetic studies.

57. The method of claim 45, wherein said Phase 1 trial comprises pharmacodynamic studies.

58. The method of claim 45, wherein said Phase 2a trial comprises establishing a dosing regimen for said drug development candidate.

59. The method of claim 45, wherein said Phase 2a trial comprises endpoint validation.

60. The method of claim 1, further comprising the steps of:
selecting said evaluated NCE as a development candidate; and
developing said development candidate.

61. The method of claim 60, wherein said developing step comprises establishing a commercial formulation for said drug development candidate.

62. The method of claim 60, wherein said developing step comprises performing Phase 2a trials in humans with said drug development candidate.

63. The method of claim 60, wherein said developing step comprises performing Phase 2b trials in humans with said drug development candidate.

64. The method of claim 60, wherein said developing step comprises establishing a protocol for Phase 3 trials in humans with said drug development candidate.

65. The method of claim 64, further comprising performing said Phase 3 trials in humans with said drug development candidate.

66. The method of claim 60, wherein said developing step comprises filing an application for registration of said drug development candidate in a foreign jurisdiction.

67. The method of claim 60, wherein said developing step comprises filing an application for registration of said drug development candidate in a foreign jurisdiction in addition with the United States.

68. The method of claim 63, further comprising applying for United States FDA approval to market said drug development candidate.

69. The method of claim 65, further comprising applying for United States FDA approval to market said drug development candidate.

70. The method of claim 1, wherein said evaluating step comprises assembling a Drug Evaluation Core Team.

71. The method of claim 70, wherein said Drug Evaluation Core Team is selected from one or more of the group consisting of scientists, clinicians, regulatory personnel, financial personnel, and marketing personnel.

72. The method of claim 1, wherein said discovering step comprises compiling a Compound Monograph.

73. The method of claim 1, wherein said evaluating step comprises assembling a Global Project Team.

74. The method of claim 73, wherein the Global Project Team is selected from one or more of the group consisting of a global medical leader, CM&C and PCD leaders, a project director, and a commercial product team leader.

75. The method of claim 1, wherein said evaluating step comprises assembling a Global Pharmaceutical Strategic Marketing Team for evaluating said NCE.

76. The method of claim 1, wherein said evaluating step comprises assembling a First-in-Human Committee for evaluating said NCE.

77. The method of claim 1, wherein said selecting step comprises determining whether a drug should enter the evaluating step within about one week of presentation by a drug discovery team.

78. The method of claim 1, wherein said evaluating step comprises defining the evaluation criteria and developing a draft clinical plan within two weeks of acceptance into the drug evaluation stage.

79. The method of claim 1, wherein said discovering step comprises assembling a Drug Discovery Committee.

80. The method of claim 79, wherein said Drug Discovery Committee is selected from one or more of the group consisting of scientists, clinicians, regulatory personnel, financial personnel, and marketing personnel.

81. The method of claim 1, wherein said discovering step comprises establishing specific criteria to define the areas of research that will be pursued for drug development.

82. The method of claim 81, wherein the areas of research for drug development are selected from one or more of the group consisting of cardiovascular disease, neurological disease, immunological disease, cancer, infectious disease, endocrine disorders, and genetic disease.

83. The method of claim 1, wherein said discovering step comprises identifying specific target diseases for drug development.

84. The method of claim 1, wherein said discovering step comprises establishing specific criteria to define target effects.

85. The method of claim 1, wherein said discovering step establishing specific criteria to define surrogate markers.

86. The method of claim 83, wherein the specific target diseases are selected from one or more of the group consisting of breast cancer, ovarian cancer, pancreatic cancer, colorectal cancer, lung cancer, prostate cancer, Parkinson's disease, Alzheimer's disease, stroke, epilepsy, schizophrenia, Huntington's disease, coronary heart disease, myocardial infarction, hypertension, arrhythmia, atherosclerosis, lupus erythematosus, scleroderma, acquired immunodeficiency syndrome, and amyloidosis, diabetes, and obesity.

87. The method of claim 81, wherein said specific criteria to define the areas of research for drug development diseases are selected from one or more of the group consisting of market potential, competitor presence, resource requirements, regulatory requirements, and patentability.

88. The method of claim 1, wherein said evaluating step comprises an evaluation of one or more of the groups consisting of validity and accuracy of the preclinical studies; the target pharmacological response; toxicity effects; and pharmacokinetic parameters including serum concentrations, absorption, distribution, and elimination.

89. The method of claim 11, wherein human cell or tissue samples are used in said cell or tissue based assays.
